(19) United States
(12) Patent Application Publication
(10) Pub. No.: US 2014/0228653 A1
(43) Pub. Date: Aug. 14, 2014

(54) DEVICE AND METHOD FOR DISPLAYING FETAL POSITIONS AND FETAL BIOLOGICAL SIGNALS USING PORTABLE TECHNOLOGY

(71) Applicant: Marianna Kiraly, San Carlos, CA (US)

(72) Inventor: Marianna Kiraly, San Carlos, CA (US)

(21) Appl. No.: 14/259,172

(22) Filed: Apr. 23, 2014

Related U.S. Application Data
(63) Continuation-in-part of application No. 13/396,233, filed on Feb. 14, 2012.
Application No. 13/396,233 is a continuation-in-part of application No. 14/068,951, filed on Oct. 31, 2013.

(51) Int. Cl. A61B 8/00; A61B 8/14; A61B 8/08; A61B 19/00; A61B 5/0478; A61B 5/0408; A61B 5/04; A61B 5/044; A61B 5/00; A61B 5/0205

(52) U.S. Cl. CPC A61B 8/466; A61B 5/0077; A61B 5/4362; A61B 5/6823; A61B 8/145; A61B 8/4494; A61B 8/0866; A61B 5/0205; A61B 8/565; A61B 5/0478; A61B 5/0408; A61B 5/04017; A61B 5/044; A61B 5/0006; A61B 8/4427; A61B 19/54; A61B 2019/5437
USPC 600/301; 600/476; 600/382; 600/443; 600/447

(57) ABSTRACT

A new, inexpensive and non-invasive fetal visualization process helps to locate fetal body parts and identify fetal positions without exposing the fetus to prolonged ultrasound irradiation. Associating location data with biological electrophysiology signal patterns and/or light absorption/reflection-related tissue-specific local fetal body composition data enables the generation of a 3D anatomical and functional map of the fetal body through the expecting mother's womb, which will be essential for long-term home monitoring of a fetus during pregnancy.

### CLAIM FOR PRIORITY AND CROSS-REFERENCE TO RELATED APPLICATIONS

This application is a continuation-in-part of U.S. Ser. No. 14/068,951 filed on Oct. 31, 2013, now pending, which in turn is a continuation-in-part of U.S. Ser. No. 13/396,233 in the name of the inventor Marianna Kiraly filed on Feb. 14, 2012, now pending, which in turn claims the priority of Provisional Patent Application Ser. No. 61/627,626, filed on Oct. 14, 2011, in the name of the inventor Marianna Kiraly. These applications are hereby expressly incorporated herein in their entirety by reference thereto.

### FIELD OF THE INVENTION

The present invention relates to basic developmental biology, portable medical obstetric procedures and devices, and more particularly to the non-invasive monitoring of the positions and certain body parts of a developing human fetus inside the mother's womb.

### BACKGROUND OF THE INVENTION

Many of the developmental disorders of childhood—cerebral palsy, epilepsy, cognitive impairment from prematurity and autism—appear to result from an interaction of complex genetic traits and environmental factors. Likewise, adult psychiatric diseases may have their origins in impaired early, even fetal development, as proposed for schizophrenia [1]. Despite major efforts, these prevalent, debilitating, life-long disorders remain biologically unexplained. Based on animal studies, the development of most types of epilepsy, cerebral palsy, autism and schizophrenia is suggested to link to neonatal seizures and various disturbances during the fetal development. The intimate connection between mother, fetus and placenta, the vast array of hormones expressed in the mother or in the placenta, or a variety of other environmental factors (injuries, drug treatments, immune responses, infections, hypoxic stress) make the targets when investigating fetal environmental disruptions that can affect fetal health. In most cases, it is already too late after birth to permanently reverse the poor health outcomes, as they are being developed early due to microenvironmental alterations. Therefore, monitoring fetal activity is crucial to prevent certain diseases.

Detecting electrical signals is generally known in the medical arts. Use of ultrasound to display a fetus or to measure its Doppler cardiogram is also generally known in the medical arts. Recording of fetal brain wave and heart signals is known in the prior art, for example in U.S. Patent No. 20020193670, U.S. Patent No. 20100274145 , U.S. Pat. No. 6,556,861 to Prichep, and in U.S. Pat. No. 7,016,722 to Prichep. The entire disclosures of these patents are expressly referred to and incorporated herein by reference thereto.

Use of a maternity belt is known in the prior art, for example in U.S. Patent Pub. No. 2007/0037483, having a publication date of Feb. 15, 2007. The entire disclosure of this patent is expressly referred to and incorporated herein by reference thereto.

Use of an optical imaging system with 3D tracking facilities is known in the prior art, for example in U.S. Patent Pub. No. 2010/0010340, having a publication date of Jan. 14, 2010. The entire disclosure of this patent is expressly referred to and incorporated herein by reference thereto.

It is a problem in the prior art to detect spontaneous brain activity in a developing fetus. As a consequence, it is also a problem in the prior art to detect signs of epilepsy or other brain injuries or disorders in a developing fetus, as in most cases these are not correlated with responses to auditory stimuli. There is accordingly a need in the prior art for a small, portable device (e.g. a smartphone-based instrument) that provides the convenience to a pregnant woman to perform such long-term measurements at home, anytime; and preferably after being trained in its use by a physician.

It is a further problem and need in the prior art to provide a portable fetal-EEG recording device that is extremely sensitive, detecting potentials of even below 1-2 microvolts, capable of detecting and recording signals over an extended period of time, and perform the steps of analyzing the recorded signals for signs of developmental brain disorders in the developing fetus.

It is also a problem in the prior art to provide visual control of the fetus, in order to determine the movement of the fetus between the time of application of the electrodes to the time of later measurements. This is intended to prevent occurrence of artifacts in the recordings. In accordance with the present invention, the brain waves of the fetus can not only be correlated to its position and activity, but also to its ECG (electrocardiogram) patterns or its Doppler-based heart rate, to better understand how its current brain activity changes during awake and sleep states.

It is a problem in the prior art to determine the position of a fetus accurately without ultrasound scanning, which is a crucial criterion for signal interpretation. Most pregnant women don't feel comfortable using an ultrasound device on themselves; therefore an alternative technique for ultrasound imaging is needed in order to be able to perform fetal electrophysiology at home, without requiring a physician's supervision. There is accordingly a need in the prior art for a non-invasive and safe way of determining and displaying fetal positions, to locate the electrophysiology biosensors to the correct positions to record fetal heart- and brain waves.

### SUMMARY OF THE INVENTION

The claimed method and portable system provides a new, inexpensive and non-invasive fetal visualization process that helps to locate fetal body parts and identify fetal positions without exposing the fetus to prolonged ultrasound irradiation. Associating location data with biological electrophysiology signal patterns and/or light absorption/ reflection-related tissue-specific local fetal body composition data enables the generation of a 3D anatomical and functional map of the fetal body through the expecting mother's womb, which will be essential for long-term home monitoring of a fetus during pregnancy.

### BRIEF DESCRIPTION OF THE DRAWINGS

FIG. 1 is a schematic view of an expectant mother together with a camera having a screen depicting the fetus within the expectant mother.

FIG. 2 is a schematic view of an expectant mother wearing a light weighted belt having a plurality of biosensor electrodes, showing connection to a portable computer equipped with a camera and imaging software.

FIG. 3 is a schematic illustration of the fetal imaging process.

FIG. 4 is a perspective view of a belt carrying an array having a plurality of light sources and detectors together with a light source and an imaging system which receives light.

FIG. 5 schematically illustrates an assembly view of the elements shown in FIGS. 1-4, separately and in use.

FIG. 6 is a schematic view of an optical detection system usable in the present invention, as shown and described in U.S. Patent Application Serial No. 2010/0010340.

FIG. 7 is a schematic view of a portable device according to the present invention, showing functional connections and output features thereof, as well as connections with an imaging device, and a fetal electrophysiology-recording device.

FIG. 8 is a schematic view of a portable device according to the present invention, showing structural features thereof and connections with a multiple light and/or ultrasound source and receiver module together with a fetal electrophysiology recording device.

FIG. 9 is a schematic flowchart of steps showing use of the invention of FIGS. 1-8.

### DETAILED DESCRIPTION OF THE INVENTION AND METHOD

The present invention, discussed in detail hereunder, relates to a portable device and a method for using the portable device to detect fetal heart rate and EEG signals, and to detect signs of normal and abnormal fetal development. The device of the present invention provides an Internet connection, and it serves as an apparatus for performing and analyzing fetal-EEG and ECG recordings in parallel with fetal visualization. Furthermore it is also capable of associating 3D maternal abdominal locations to fetal tissue-specific optical features (light absorption, reflection), and matches fetal body anatomy with functional organ-specific electric activity patterns.

In many countries, practically all pregnant women undergo routine obstetric ultrasound (US) examinations, once or several times during pregnancy. Usually, one of the examinations is done at 16 to 22 gestational weeks for detection of fetal anomalies. At this time of pregnancy, the migration of neurons into the fetal neocortex is not finished [2] and concerns have been expressed that ultrasound might disturb this process [3]. In a Norwegian study [4], a cohort of pregnant women were divided in two groups. Half of the mothers had real ultrasound scanning during pregnancy while the others had a sham investigation. When the children were examined after birth there was significant excess of left-handedness only in the group exposed to real ultrasonography. In addition, recent in vitro and in vivo results demonstrate that US can be used to modulate action potential firing and synaptic transmission [5-11]. Consequently, there has been an emerging need to develop a new fetal visualization technique as an alternative to ultrasound scanning that may be suitable for everyday home use for monitoring high-risk pregnancies.

Technologies which can be used in the present invention, and which are commercially known and available for use, are known in the art and samples of these are as follows. Portable computer software capable of overlaying computer graphics on the real world (e.g. a real-time camera image) are broadly known and referred to as Augmented Reality (AR) in prior arts. Examples would be known to any one skilled in the 3D imaging arts and especially the patents classified in US patent class 345/633 (real-time Augmented Reality), such as Nokia's U.S. Patent Application 20120075341 published on Mar. 29, 2012. The type of electrodes and method of use feasible for the present invention are known, for example in U.S. Pat. No. 6,162,101 issued on Sep. 3, 1998 to Fisher and Iversen; U.S. Pat. No. 6,024,702 issued on Feb. 3, 1997 to Iversen; U.S. Pat. No. 5,961,909 issued on Sep. 3, 1997 to Iverson; U.S. Pat. No. 5,902,236 issued on Sep. 3, 1997 to Iversen; as well as in other patent documents. The possibility of recording spontaneous electrical brain and heart activity of a fetus in utero has also been published [12, 13].

Hand-held optical probes utilizing near-infrared (NIR) 3D imaging for diagnostic purposes have also been known in prior art, for example from U.S. Patent Application 2010/0010340 A1 published on Jan. 14, 2010. In addition, it has been reported that different biological tissue types show differences in their light absorption and reflection properties [14]. Accordingly, for example fetal tissue types that are rich in fat and water (such as the brain) have a maximum absorption peak at the wavelenth of ˜930 nm; whereas umbilical arteries and veins have their highest absorption peaks at ˜410 nm and ˜580 nm. These differences have been utilized for agricultural purposes (quality control of food items) or body composition screening (for example, U.S. Pat. No. 7,711,411 B2; issued on May 4, 2010), and in medical device arts to screen intracranial bleeding in head trauma patients (for example, U.S. Pat. No. 8,060,189), or placental circulation [15]. However, it has never been proposed as an indirect alternative of ultrasound, feasible for fetal monitoring via measuring the body composition of the fetus.

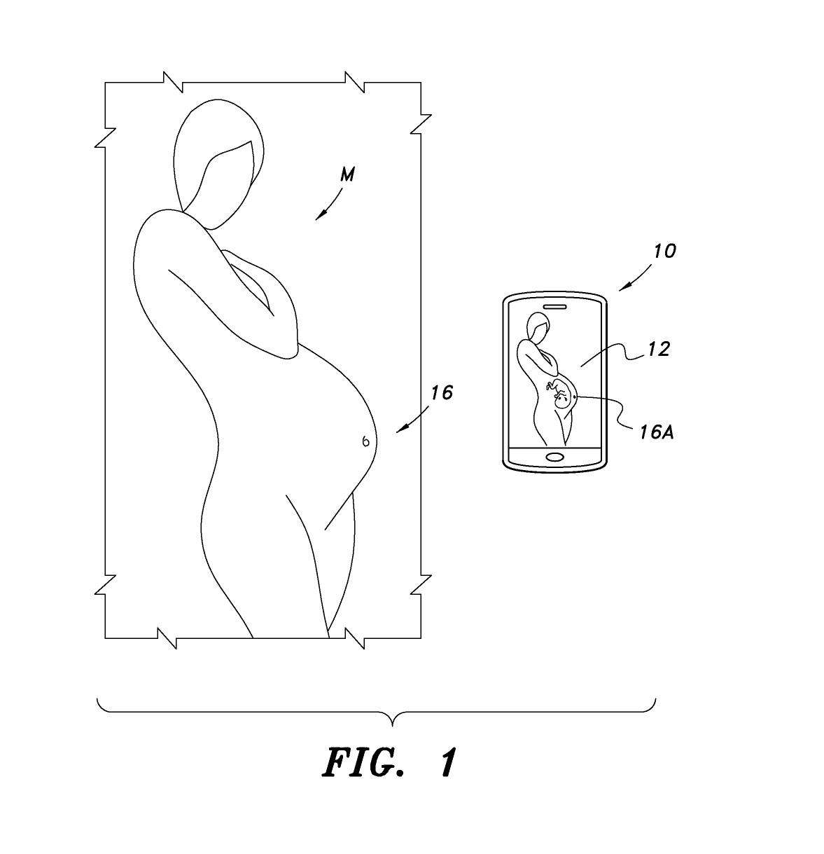

FIG. 1 is a schematic view of an expectant mother M having an abdomen region 16 together with a portable computer device 10 having a camera with a display screen 12 depicting a fetus 16A within the expectant mother. That is, FIG. 1 is a simple schematic demonstration of the portable computer device 10 equipped with the camera and an imaging software. The imaging software algorhythm is capable of performing real-time image processing, and identifying the shape of a pregnant woman's abdomen based on its curving, the umbilicus or any calibrated artificial markers attached to the abdomen. Once the identification has happened, it is capable of overlaying the mother's abdomen with a computer image, animation, ultrasound picture or real-time ultrasound video of the fetus on the computer display, using certain given parameters and formulas (e.g. weeks of pregnancy, size of the abdomen in centimeters, estimated fetal position).

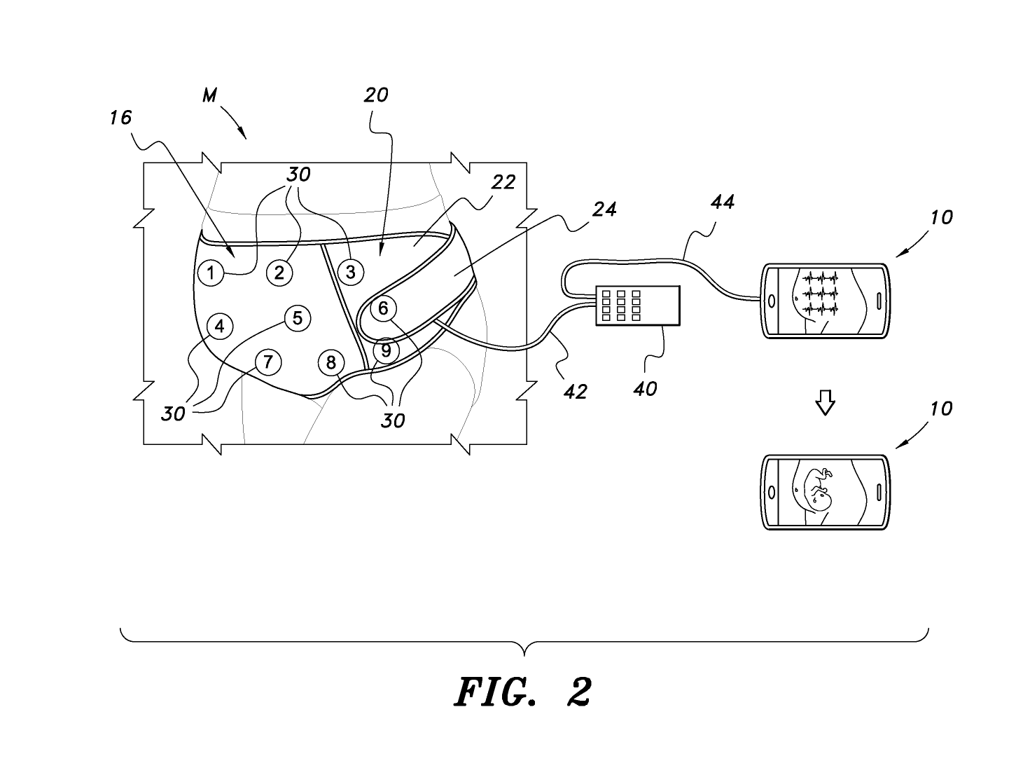

FIG. 2 is a schematic view of the expectant mother M wearing a light weighted belt 20 having a plurality of biosensor electrodes 30 (which have circled numerals thereon 1-9), showing connection thereof to a portable amplifier-filter-digitizer module 40, and in turn to a portable computer 10 equipped with a camera and imaging software. The belt 20 includes a main portion 22 and a fastening strap portion 24. The portable amplifier-filter-digitizer module 40 is connected to the electrodes 30 by a cord 42, and the output of the portable amplifier-filter-digitizer module 40 is sent via a cord 44 to the portable computer 10.

In FIG. 2, the light weighted belt 20 is comfortably attached to a pregnant woman's abdomen 16, overlying the head and chest of the fetus. The light weighted belt 20 has the multiple biosensor electrodes 30 embedded in its inner surface, connected to the portable amplifier-filter-digitizer module 40 and the portable computer 10 which is equipped with a camera and imaging software. The biosensors 30 transmit fetal biological signals to a portable computer via wires, Bluetooth, GSM or Wi-Fi connection, and the computer software calculates and projects an approximate fetal position on the maternal abdomen, based on the relative amplitudes of the organ-specific signal (e.g. rhythmic heartbeat) patterns.

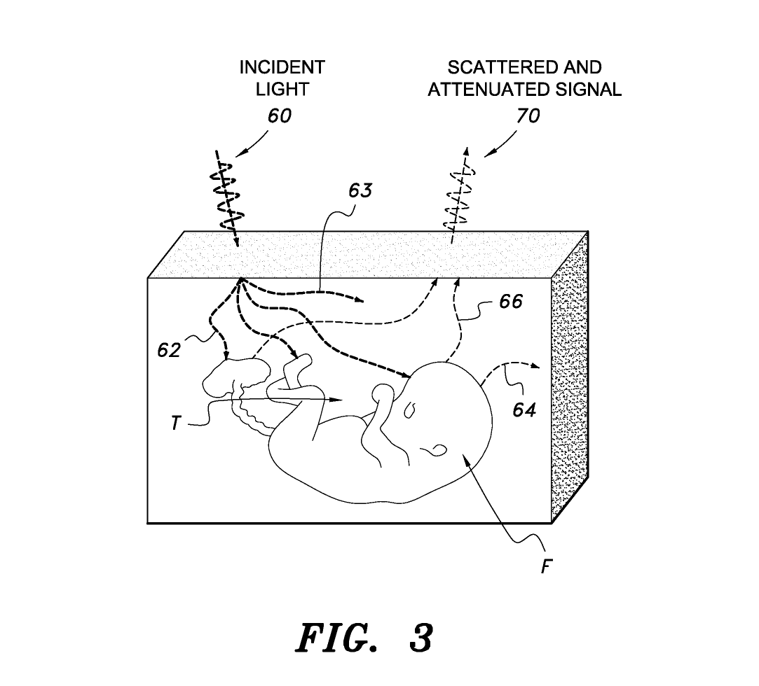

FIG. 3 is a schematic illustration of a fetal imaging process. FIG. 3 illustrates the basic optical imaging mechanism. The light sources 60 generate multiple NIR, visible or infrared rays of given wavelenghts (e.g. 938 nm or 580 nm wavelenghts to measure fetal brain tissue absorption; whereas the 730 nm and 850 nm wavelenght pair is scattered by brain tissue and is mainly absorbed by oxy- and deoxy-Hemoglobin) sequentially or simultaneously. The light rays are launched on the maternal abdomen and penetrate in the tissue up to 4-5 cm depth or even deeper. Corresponding light detectors characterize the intensity patterns and time-dependent parameters (e.g. phase, delay) of the reflected/ absorbed light rays. Fetal tissue composition is characterized by a variety of optical tissue properties at different wavelenghts (e.g. absorption, scattering, scattering function), or their derivatives (e.g. anisotrophy, real refractive index, reduced scattering). Here, incoming rays are schematically depicted as rays 62, 63, and scattered and/or reflected rays are schematically depicted as rays 64, 66. The fetus F is in the target region T of the incident light 60, and becomes a reflected, scattered or attenuated light signal 70, modified in a tissue-specific manner by the fetal organs before reaching the detectors.

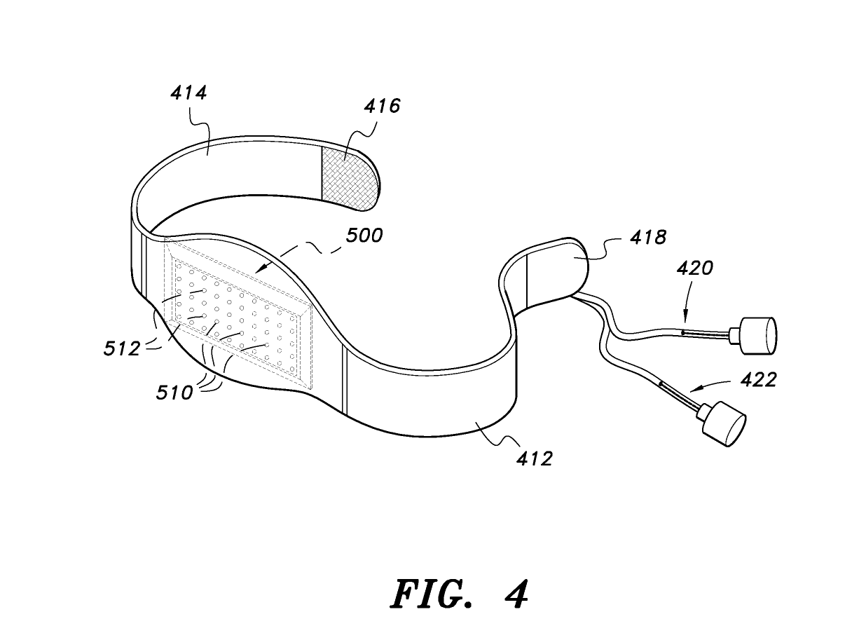

FIG. 4 is a perspective view of a belt corresponding to the belt 20 described above, carrying an array 500 having a plurality of light sources 510 and a plurality of detectors 512, optionally combined with an external light source 422 and an imaging system 420 which receives light. In FIG. 4, the sources 510 alternative vertically and horizontally with the detectors 512. As shown in FIG. 4, the belt includes a main body portion 412, 414 and connecting straps 416, 418. The straps 416, 418 can be secured by hook-and-loop fastener elements, for example, or by any other type of known fastening means.

FIG. 4 illustrates the structure of light transmitters 510 and detectors 512 in the pregnancy monitoring belt. The light sources might be individual LED lights or lasers that can be operated in a programmed manner via a portable computer, or distributed from one central light source via optic cables that terminate in the inner surface of the belt. In another embodiment, the belt is light protected on its outside surface, and is equipped with a thermoregulator, preventing the heat to dissipate towards the mother's abdomen and the fetus. The detectors transmit the optical output data to a CCD Imaging system or other type of processig unit.

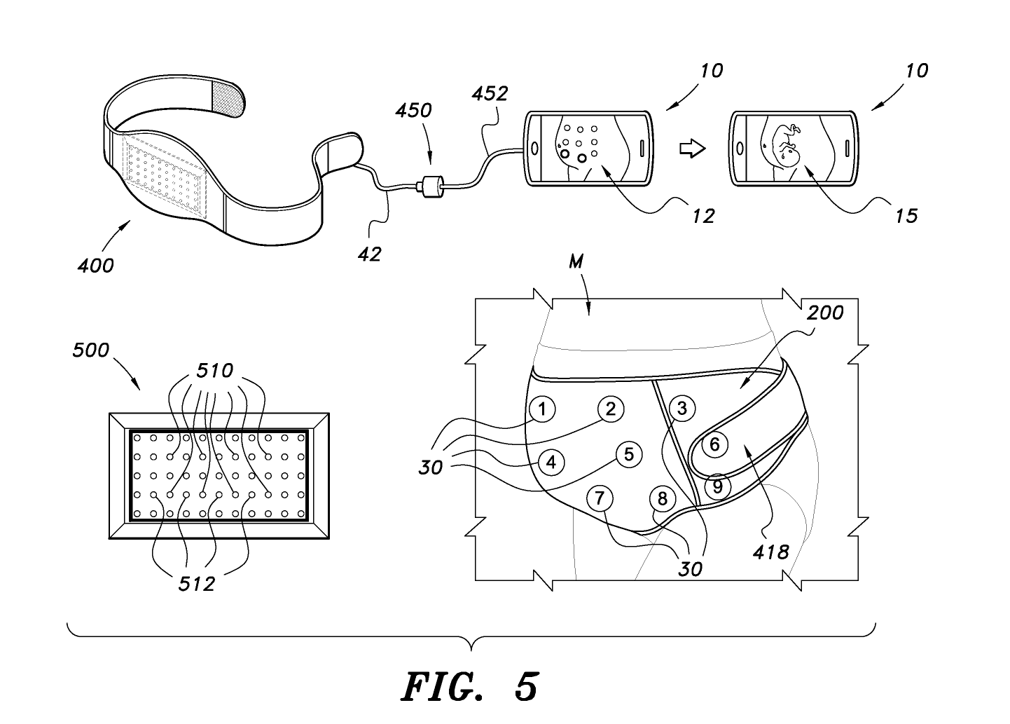

FIG. 5 schematically illustrates an assembly view of the elements shown in FIGS. 1-4, separately and in use. The belt of FIG. 4 is indicated by numeral 400 in FIG. 5, and also shows the array 500 having the source-detector distribution pattern 510, 512 on the inner surface of the biosensor belt 400. Each source is surrounded by multiple detectors, and different sets of separately operated emitters and signal-receivers (e.g. miniature ultrasound transducers and receivers; or 580 nm/938 nm light sources and sensors) can be positioned in close proximity to one another, within the same belt. This way, it is possible to perform a complex, multi-channel fetal tissue composition and bodypart-distance analysis simultaneously or sequentially. The collected signals can be first fiber-transmitted 42 into a signal processing unit (CCD camera & intensifier-filter unit) 450, then to a portable computer equipped with a camera and imaging software, via conventional wires 452 or wireless connection such as Wi-Fi, GSM or Bluetooth communication. The imaging software generates a 3D data map from the inputs, and projects it on the maternal abdomen overlaying a virtual fetal body shape. The mother M carries the belt indicated here as belt 200, using fastener elements 418, with the sensors 30 schematically depicted in circled numerals (1-9) for the sake of clarity.

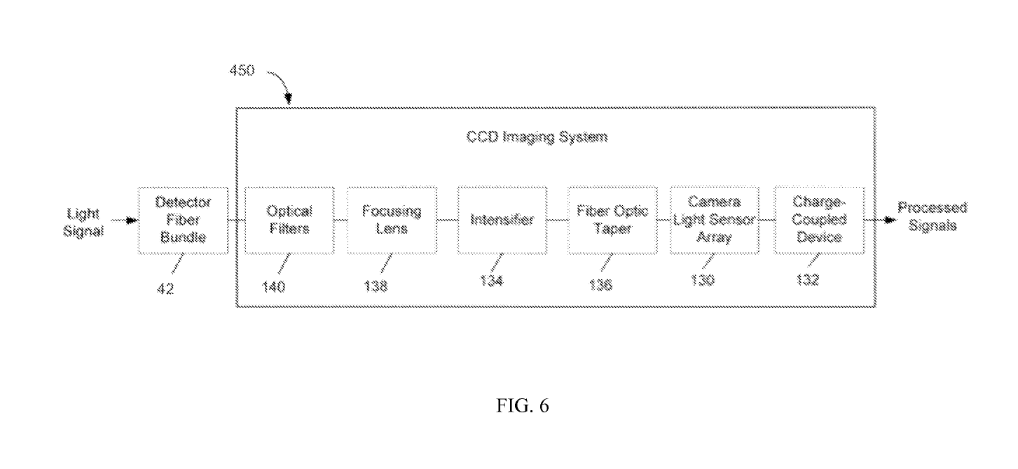

FIG. 6 is a schematic view of an optical detection system usable in the present invention, as shown and described in U.S. Patent Application Serial No. 2010/0010340. The CCD imaging module or system numbered as 450 in FIG. 5, as described in U.S. Patent Application 2010/0010340 A1 has been incorporated herein by reference.

Specifically, FIG. 6 includes a light signal entering a detector fiber bundle 42 as shown in FIG. 5, which feeds the CCD imaging system 450. The system 450 has optical fibers 140, a focusing lens 138, an intensifier 134, a fiber optic taper 136, a camera light source array 130, and a charge-coupled device 132.

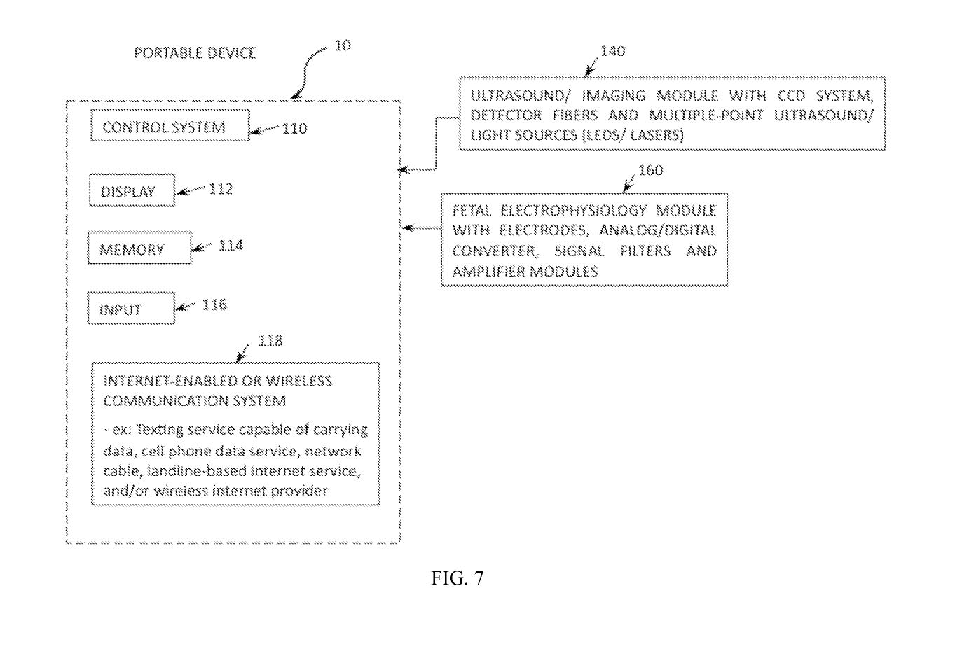

FIG. 7 is a schematic view of a portable device 10 according to the present invention as demonstrated in FIGS. 1-2, showing functional connections and output features thereof, as well as connections with a fetal imaging system as presented in FIG. 5, and a fetal electrophysiology-recording system as shown in FIG. 2. FIG. 7 is a schematic view of the portable device 10 according to the present invention, showing structural features thereof and having connections with a portable ultrasound and/or imaging module 140. The module 140 is equipped with a data processing unit, a CCD system, detector fibers and multiple-point ultrasound and/or light sources such as LEDs or lasers. The device is also connected to a module 160 which is a portable fetal electrophysiology module with attached electrodes, analog-digital converter, signal filters and an amplifier unit.

As seen in FIG. 7, the portable device 10 is shown in dashed outline, and preferably includes a control system 110, a display 112, a memory 114, an input means 116 (such as a touch pad, a keyboard, a mouse, or other input devices), and an internet-enabled or wireless communication system 118. The internet-enabled or wireless communication system 118 can be of a type already known in smartphone technologies, or it can be a custom-built portable device within the ambit of skill of any one having skill in the smartphone arts. The elements 110, 112, 114, and 116 can all be types which are present in existing smartphone technologies, or can be custom made within the ambit of skill of any one having skill in the smartphone arts and/or the smartphone application programming arts.

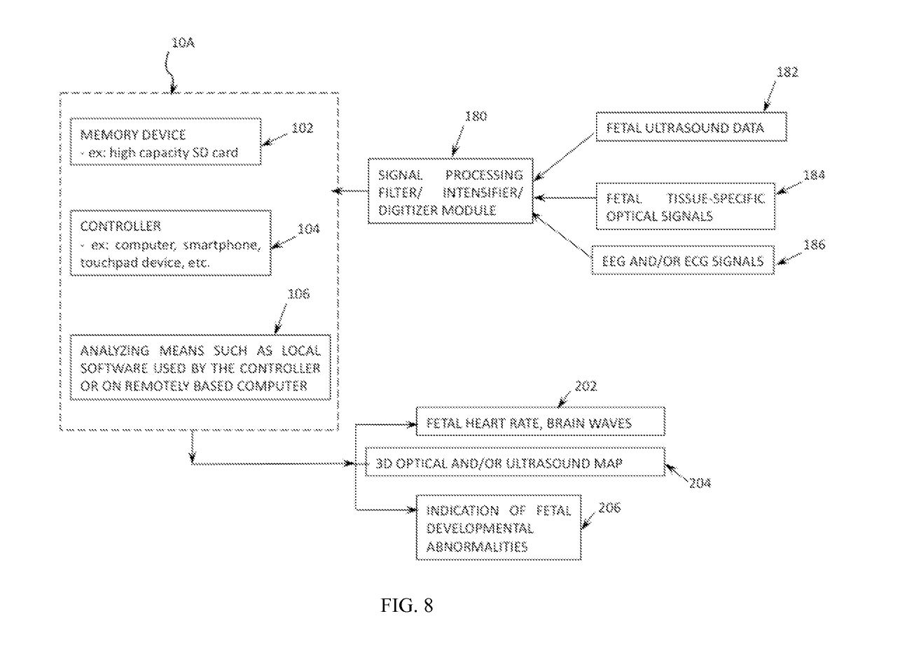

FIG. 8 is a schematic view of a portable device 10A. Specifically, FIG. 8 shows the portable device 10A receiving input signals from a signal processing/filtering/amplifying unit 180 which in turn is supplied with input signals representing fetal ultrasound data 182, fetal tissue-specific optical signals 184, and EEG and/or ECG signals 186. Instead of or in addition to ultrasound data, the element 182 can represent signals from the light sources and signal detecting device shown in above-described FIGS. 3-6.

FIG. 8 also shows an output 202 from the device 10A which represents fetal heart rate and brain waves, an output 204 which represents 3D optical and/or ultrasound map, and an output 206 which represents an indication of fetal developmental abnormalities.

The element 186 can be a fetal-EEG and/or ECG recording device which records EEG and/or ECG signals from electrodes. The portable device 10A can be similar or identical to the portable device 10 shown and discussed hereinabove, or it can be a variation of that device.

The portable device 10A includes a memory device 102 which can, for example, be a high capacity SD card or other type of memory device. The portable device 10A also includes a controller 104 which can, for example, be a computer or computer chip, a smartphone, smart touchpad device having computer techology, etc.

The portable device 10A also includes an analyzing function means 106 such as local software used by the controller 104, or else supplies data to a remotely based computer for software analysis using the internet or cell phone technology.

The portable device 10A provides outputs, which can include fetal heart rate, noise and artifact filtered EEG, ECG and/or integrated EEG signals 202, 3D optical and fetal body composition map, and an indication of fetal developmental abnormalities such as intrauterine seizures or other abnormal brain- or heart activity, or abnormal durations of sleep and awake states 206. These signals can be obtained using the software represented by block 106.

The detection and determination of normal and abnormal human fetal brain activity is an evolving field. It is anticipated that future discoveries may be made in this evolving field, and it is contemplated that the results of such discoveries can be used in the indication of abnormal fetal development 206.

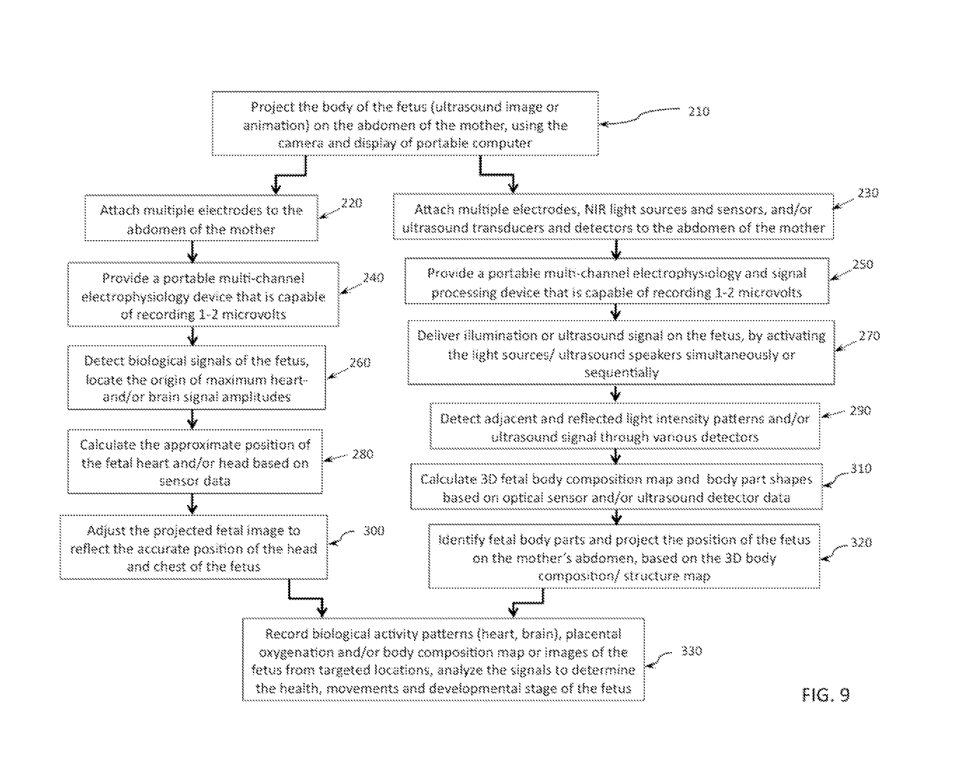

FIG. 9 is a schematic flowchart of steps showing use of the invention of FIGS. 1-8. Here, step 210 is use of a portable device with a camera and imaging software, where the software operates to perform real-time identifying of the shape of a pregnant woman's abdomen and projecting the approximate position and size of the fetus (animation, computer image or ultrasound picture) onto it. Then at step 220, an electrode sheet is applied to the mother's abdominal region over the head and/or heart of the fetus.

In step 240, a portable multi-channel electrophysiology recording is connected to the electrodes. Step 260 is using the portable device 10 or 10A to record the brain and/or heart activity of the fetus (using the signals received from the electrode or electrode sheet) for extended time periods. In step 280, the organ-specific electric signals are associated with 3D locations, and the electrode position where the maximum amplitudes of fetal ECG signal appears is matched with a virtual heart position, and the electrode positions where the maximum amplitudes of fetal brain-wave signals appear are correlated to fetal head positions.

In step 300, the fetal image projected in step 210 is adjusted to the 3D signal map determined in step 280. Finally, in step 330 the portable device 10 or 10A records and stores the registered data and images, analyzes the signals and determines the health status, movements and developmental stage of the fetus.

In another embodiment, step 230 is attaching multiple electrodes, light sources and sensors, and/or ultrasound transducers and detectors to the abdomen of the mother. In step 250, a portable multi-channel electrophysiology recording and signal processing device is connected to the electrodes. Step 270 is using the portable ultrasound and/or imaging module with CCD system, detector fibers and multiple-point ultrasound transducers and/or light sources to launch ultrasound signals and/or light rays of specific wavelengths on the abdomen of the mother in a simultaneous or sequential manner. In step 290, the reflected bodypart- or object-distance-specific ultrasound signal, and/or the tissue-specific light intensity, and/or functional electric signals are associated with 3D locations.

In step 310, portable device 10 or 10A generates a 3D fetal body composition and/or functional tissue-specific electric signal map and/or ultrasound image map based on the optical, ultrasound and electric input data. In step 320, the fetal image projected in step 210 is adjusted to the 3D signal map determined in step 320. Finally, in step 330 the portable device 10 or 10A records and stores the registered data and images, analyzes the signals and determines the health status, movements and developmental stage of the fetus according to the previously generated 3D map.

Also in FIG. 9 the step 330 is analyzing the above-mentioned detected signals using software in a real time manner or at a later time using the portable device 10 or 10A to communicate results (raw and/or analyzed data) using telecommunication means as discussed hereinabove (e.g. internet, cell phone transmissions, etc.) to an obstetrician or other professionals at any time. The Step 330 also is contemplated to include transmitting stored data saved over a relatively long period of time, and having that data analyzed by remote software, by an obstetrician, or by other professionals at any time.

Optionally, step 330 may include using the portable system to take pictures and/or videos and/or sound files of the baby to send to relatives, friends, and/or medical professionals, and/or to provide a continuous stream of video for webcam or videoconferencing purposes. In another embodiment, the imaging software is further capable of detecting fetal motions, and adjusting the 3D data map automatically, according to the newly recorded electric signal-maximum or body composition data coordinates, or reflected ultrasound signals.

The invention being thus described, it will be evident that the same may be varied in many ways. Such variations are not to be regarded as a departure from the spirit and scope of the invention and all such modifications are intended to be included within the scope of the claims.

### REFERENCES

1. Meyer U, Yee B K, Feldon J.: The neurodevelopmental impact of prenatal infections at different times of pregnancy: the earlier the worse? Neuroscientist. 2007 June; 13(3):241-56.
2. Meyer G (2001) Human neocortical development: the importance of embryonic and early fetal events. (Translated from eng) Neuroscientist 7(4):303-314 (in eng).
3. Mole R (1986) Possible hazards of imaging and Doppler ultrasound in obstetrics. (Translated from eng) Birth 13 Supp1:23-33 suppl (in eng).
4. Salvesen K A, et al. (1992) Routine ultrasonography in utero and subsequent vision and hearing at primary school age. (Translated from eng) Ultrasound Obstet Gynecol 2(4):243-244, 245-247 (in eng).
5. Legon W, Rowlands A, Opitz A, Sato T F, & Tyler W J (2012) Pulsed ultrasound differentially stimulates somatosensory circuits in humans as indicated by EEG and FMRI. (Translated from eng) PLoS One 7(12):e51177 (in eng).
6. Tufail Y, Yoshihiro A, Pati S, Li M M, & Tyler W J (2011) Ultrasonic neuromodulation by brain stimulation with transcranial ultrasound. (Translated from eng) Nat Protoc 6(9):1453-1470 (in eng).
7. Tufail Y, et al. (2010) Transcranial pulsed ultrasound stimulates intact brain circuits. (Translated from eng) Neuron 66(5):681-694 (in eng).
8. Tyler W J (2011) Noninvasive neuromodulation with ultrasound? A continuum mechanics hypothesis. (Translated from eng) Neuroscientist 17(1):25-36 (in eng).
9. Tyler W J, et al. (2008) Remote excitation of neuronal circuits using low-intensity, low-frequency ultrasound. (Translated from eng) PLoS One 3(10):e3511 (in eng).
10. Yoo S S, et al. (2011) Focused ultrasound modulates region-specific brain activity. (Translated from eng) Neuroimage 56(3):1267-1275 (in eng).
11. Rinaldi P C, Jones J P, Reines F, & Price L R (1991) Modification by focused ultrasound pulses of electrically evoked responses from an in vitro hippocampal preparation. (Translated from eng) Brain Res 558(1):36-42 (in eng).
12. Khandoker, A. H.; Kimura, Y.; Palaniswami, M.; Marusic, S.: Identifying fetal heart anomalies using fetal ECG and Doppler cardiogram signals. Computing in Cardiology, 2010 September; 891-4.
13. Lindsley D B.: Heart and brain potentials of human fetuses in utero. Am J Psychol. 1987 Fall-Winter; 100(3-4):641-6.
14. Jacques, S L: Optical properties of biological tissues: a review. Phys. Med. Biol. 2013, 58:R37-R61
15. Kakogawa, J. and Kanayama, N.: Application of near-infrared spectroscopy for the evaluation of placental oxygenation. The Open Medical Devices Journal, 2012, 4, 22-27

What is claimed is:

1. A method of virtually projecting a computer image of a fetus (2D or 3D animation, ultrasound or drawing) on a pregnant woman's abdomen, comprising the steps of:
(a) providing a portable computer equipped with camera, imaging software and a visual display;
(b) providing an “Augmented Reality” software capable of performing real-time image processing, and identifying the shape of a pregnant woman's abdomen based on its curving, the umbilicus or any calibrated artificial markers attached to the abdomen;
(c) providing a 2D or 3D computer image of the fetus (previously captured or real-time ultrasound image, animation or drawing);
(d) having the portable computer calculate an approximate projection of the fetus, based on given parameters and formulas such as correlations between fetal size and abdominal size, last known fetal position, weeks of pregnancy
(e) having the “Augmented Reality” software overlay the computer image of the fetus with the abdomen of the pregnant woman

2. The method of recording as claimed in claim 1, further comprising the step of adjusting the projected fetal image to a 3D electrical signal map recorded through the abdominal wall via multiple electrodes, so that the location of maximum organotypic (head or heart) signal amplitudes correspond to the appropriate fetal organ (heart/chest/head) visualization.

3. The method of recording as claimed in claim 1, further comprising the step of adjusting the projected fetal image to 3D ultrasound signals reflected by the fetus through the mother's abdominal wall in real-time manner, generated and detected by one or more transducers attached to the abdomen.

4. The method of recording as claimed in claim 1, further comprising the step of adjusting the projected fetal image to a 3D infrared, near-infrared, or visual light signal map generated and detected via multiple sources and sensors attached to the abdominal wall, so that the location of maximum/minimum tissue- and organ-specific (brain, placenta, umbilical chord) optical signal intensities (absorbed, scattered or reflected lights of different wavelengths) correspond to the appropriate fetal body-part visualization.

5. The method of recording as claimed in claim 1, further comprising the step of adjusting the projected fetal image to reflect fetal movements, detected as changes in locally detected ultrasound signal, light intensity or organ-specific electric signal.

6. The method of recording as claimed in claim 1, further comprising the step of adjusting the position of the attached abdominal 3D sensor sheet to cover targeted body parts of the fetus, according to the adjusted image projection of the fetus.

7. The method of recording as claimed in claim 1, further comprising the step of transmitting any of the images, videos or recordings via the internet.

8. The method of recording as claimed in claim 1, further comprising the step of comparing the fetal ultrasound/EEG/ECG/body composition data to reference fetal ultrasound/EEG/ECG/optical signals from a control group or the same fetus' own previously recorded data to determine one of an abnormality and normality ultrasound/EEG/ECG/optical signals of the fetus being monitored.

9. The method of recording as claimed in claim 1, further comprising the step of comparing the fetal ECG signals to the mother's ECG signals, and extracting the maternal signals from the fetal signals.

10. A portable fetal monitoring device for recording biological signals from a fetus in utero, comprising:
(a) an “Augmented Reality” software capable of performing real-time camera image processing and identifying the shape of a pregnant woman's abdomen based on its curving, the umbilicus or any calibrated artificial markers attached to the abdomen; calculate an approximate projection of the fetus, based on given parameters and formulas such as correlations between fetal size and abdominal size, last known fetal position, weeks of pregnancy; and overlaying a random computer image of the fetus with the abdomen of the pregnant woman
(b) an array of sensor electrodes adapted to be placed on the mother's abdomen for detecting electrical activity of a fetus;
(c) an amplifier-filter module connected to the array of sensors to amplify the spontaneous brain activity of the fetus detected by the biosensor electrodes;
(d) an analog/digital converter converting the analog data to digital data;
(e) a portable computer-based quantitative analysis software capable of improving a signal to noise ratio of the digitized spontaneous brain activity data and analyzing the data;
(f) a display to real-time demonstrate the raw data and the results of the analysis as an indication of a status of the fetus; and
(g) portable computer-based memory to store the data, and being capable of outputting said data for transmission to an external device or network.

11. The portable fetal monitoring device as claimed in claim 10, wherein the array of sensor electrodes is coupled with at least one sensor electrode placed on the mother!s chest, to record maternal ECG signals.

12. The portable fetal monitoring device as claimed in claim 10, wherein the array of sensor electrodes is coupled with multiple light sources (LEDs or lasers) and detectors in the same portable platform.

13. The portable fetal monitoring device as claimed in claim 10, wherein the array of sensor electrodes is coupled with multiple ultrasound transducers and detectors in the same portable platform.
